(19) United States
(12) Patent Application Publication
(10) Pub. No.: US 2002/0160034 A1
(43) Pub. Date: Oct. 31, 2002

(54) Use of occlusion device for the local delivery of biologically active DNA therapeutic compounds for treating aneurysms and use therefor

(76) Inventors: Luc Levesque, Boucherville (CA); Jean Raymond, Montreal (CA); Guy Leclerc, Rosemere (CA); Francis Gauthier, Montreal (CA)

Correspondence Address: NIXON PEABODY LLP, ATTENTION: DAVID RESNICK, 101 FEDERAL STREET, BOSTON, MA, 02110, US

(21) Appl. No.: 10/081,734

(22) Filed: Feb. 22, 2002

Related U.S. Application Data
(60) Provisional application No. 60/270,606, filed on Feb. 23, 2001.

(51) Int. Cl. A61K 48/00

(52) U.S. Cl. 424/426; 514/44

(57) ABSTRACT

The invention relates to a method for the sustained endovascular treatment of aneurysms, such as intracranial or for closing any body lumen, such as vascular lumen or other using an occlusion device for the local delivery of biologically active DNA therapeutic molecules. The invention also relates to a method for a rapid preparation of the artificial occlusion device to be coated shortly before or during the clinical procedure. A subsequent step in the method of treatment involves introducing at an aneurysm site or inside a vessel a slow-releasing, biologically active DNA molecule-leaching device. The device releases a biologically active DNA molecule at the aneurysm site for stimulating neointima formation and for increasing neointima thickness. The neointima formation fills the aneurysm and the biologically active DNA molecule released in the aneurysm is absorbed by surrounding tissues of the aneurysm for providing long-term treatment of the aneurysm preventing recanalisation.

### BACKGROUND OF THE INVENTION

(a) Field of the Invention

The invention relates to a method for reducing or blocking the rate of blood flow in a vessel and more particularly to a method for treating hypervascular lesions or aneurysms, such as intracranial or for closing any body lumen, such as vascular lumen or other using an occlusion device for the local delivery of biologically active DNA therapeutic molecules.

(b) Description of Prior Art

Direct surgical clipping has been used for the treatment of most intracranial aneurysms. However, surgical difficulties and related morbidity with certain aneurysms have stimulated the development of endovascular procedures. Despite the favorable results of endovascular platinum coil treatment in acutely ruptured aneurysms, neck remnants and recurrences are frequent and may compromise the long-term success of this treatment modality. This mechanical failure of the device of the prior art is not surprising and coils alone without efficient healing mechanisms may not be strong enough to counteract the continuous repetitive force of the abnormal blood flow that often remains following incomplete endovascular treatment. The mechanism of surgical clipping directly apposes the vessel wall, leading to rapid “primary healing”. By opposition, following endovascular treatment, the wound margins are separated by coils and healing depends on fibrous replacement of clot between coils and growth of a neointima at the coil—parent vessel interface. There is a general pattern of wound healing in the vessel wall, which occurs following a wide variety of traumatic or pathological conditions. These mechanisms are also involved in repairing experimental aneurysms. In vivo studies suggest that healing of experimental aneurysms involves coagulation, inflammation, cellular migration, proliferation, and matrix secretion with the formation of a neointima at the neck of treated aneurysms. These healing factors are opposed by “recanalization”, a process that involves rapid endothelialization of slit like spaces between coils and the aneurysm wall.

Intracranial aneurysms can be treated by four different principles:

A) Surgical clipping permits closure of the aneurismal neck from the outside, with close apposition of the edges of the “wound” and satisfactory healing, but necessitates craniotomy and dissection at the base of the brain.

B) Parent vessel occlusion consisting of occlusion of the vessel along with the aneurysm or with the intent to decrease blood flow to the aneurysm, is possible only in certain anatomical sites, and in the presence of an adequate collateral circulation.

C) Parent vessel stenting is a new possibility but is currently technically feasible only in proximal vessels or in extra cranial aneurysms such as the aorta (WO 98/12990 and WO 93/08767).

D) Selective endosaccular occlusion of the aneurysm is currently the most frequently used method of endovascular treatment. This method can be performed with three (3) different types of material:

1) Liquid or fluid agents which polymerize inside the aneurysm or immediately before exiting the catheter; this strategy has never been routinely used because of the fear of cerebral embolization;

2) Detachable balloons have been introduced by Serbinenko, Romodanov and Scheghlov and have more frequently been used between 1978 and 1990. The expertise necessary for using these devices was difficult to master; these devices led to a high incidence of inadvertent aneurysm rupture and were also plagued with a high incidence of recurrences; and

3) Micro coils; these metallic devices became popular with the Guglielmi Detachable Coil system, which permitted to reposition the coil and detach it only when it was felt to be in a satisfactory position. The availability of this system has greatly increased the use of the endovascular route in the treatment of intracranial aneurysms. This device is much safer to use than detachable balloons, free coils, or polymeric embolic agents. The main advantage of soft coils compared to detachable balloons is the fact that they will conform to the shape of the aneurysms. However, recurrences after a few months are frequent and this fear of recurrences is currently the major drawback of the technique and the most important argument against a more widespread clinical application.

It would be highly desirable to be provided with a device for treating aneurysms, which could prevent recanalization and stimulate neointima formation at the neck and within the treated aneurysm for improving long-term results of endovascular treatment.

### SUMMARY OF THE INVENTION

One aim of the present invention is to provide local delivery of biologically active DNA molecules into an aneurismal sac that will stimulate and/or increase neointima formation of treated aneurysms for improving long-term results of endovascular treatment.

Another aim of the present invention is to provide local delivery of biologically active DNA molecules into the aneurismal sac that will prevent and/or inhibit recanalization of treated aneurysms for improving long-term results of endovascular treatment.

Another aim of the present invention is to provide a rapid loading process of a biologically active DNA molecule on the surface of a leaching artificial occlusion device to prevent and/or inhibit recanalization and stimulate and/or increase neointima formation within the aneurysm and at the neck of treated aneurysm for improving long-term results of endovascular treatment.

In accordance with the present invention, there is provided a method for reducing or blocking blood flow in a vessel, said method comprising the step of introducing at a desired site in the vessel a slow-releasing, biologically active DNA molecule-leaching device, said device releasing a biologically active DNA molecule at the site for stimulating neointima formation and increasing neointima thickness, said neointima formation reducing or blocking the blood flow in the vessel, said biologically active DNA molecule released at the site is absorbed by surrounding tissues of the vessel for providing long-term vascular reduction or obstruction of the blood flow in the vessel.

Still in accordance with the present invention, there is provided a method for sustained vascular occlusion of a blood vessel, said method comprising the step of introducing at a site in the vessel a slow-releasing, biologically active DNA molecule-leaching device, said device releasing a biologically active DNA molecule at the site for stimulating neointima formation and increasing neointima thickness, said neointima formation filling the vessel, said biologically active DNA molecule released at the site is absorbed by surrounding tissues of the vessel for providing long-term vascular occlusion of the blood vessel and preventing recanalisation.

Further in accordance with the present invention, there is provided a method for sustained treatment of a hypervascular lesion, said method comprising the step of introducing in the vessel feeding the lesion a slow-releasing, biologically active DNA molecule-leaching device, said device releasing a biologically active DNA molecule in the lesion for stimulating neointima formation and increasing neointima thickness, said neointima formation reducing or blocking blood flow in the vessel at the lesion, said biologically active DNA molecule released at the lesion is absorbed by surrounding tissues of the vessel for providing long-term vascular reduction of blocking of the blood flow in the vessel.

Still in accordance with the present invention, there is provided a method for preparing a DNA leaching artificial occlusion device, said method comprising the step of providing a solution of an HPLC-purified DNA and dipping an artificial occlusion device in said solution for adsorbing DNA onto the occlusion device in such a manner that said DNA leaches from said occlusion device. The HPLC-purified DNA may transiently contain a dimethoxytrityl (DMT) moiety.

In one embodiment of the invention the method further comprises before the step of dipping the occlusion device in the solution, a step of desalting the HPLC-purified DNA. The solution of HPLC-purified DNA is preferably heated above 65° C. before dipping the occlusion device therein.

In accordance with the present invention there is provided a device for treating aneurysms, which could prevent and/or inhibit recanalization and stimulate and/or increase neointima formation at the neck and within treated aneurysms for improving long-term results of endovascular treatment.

In accordance with the present invention, the biologically active DNA molecule can either be a radioactive DNA molecule, an antisense DNA molecule to inhibit the expression of genes or a DNA plasmid that can induce gene expression in adjacent tissues surrounding the endovascular device for stimulating cell proliferation.

Still in accordance with the present invention, there is provided a rapid-loading process for depositing a biologically active DNA molecule onto the artificial occlusion device. The method comprises the step of immersing the coil into a solution containing the biologically active DNA molecule under suitable conditions for loading of the biologically active DNA molecule.

The biologically active DNA molecule is preferably a radioactive DNA molecule, which may consist of two elements: a radioisotope responsible for emitting the radiation and a carrier molecule covalently link to the radioisotope.

Preferably the radioisotope comprises a emitter. Preferred β-emitters are selected from the group consisting of Antimony-124, Cesium-134, Cesium-137, Calcium-45, Calcium-47, Cerium 141, Chlorine-36, Cobalt-60, Europium-152, Gold-198, Hafnium-181, Holmiun-166, Iodine-131, Iridium-192, Iron-59, Lutetium-177, Mercury-203, Neodymium-147, Nickel-63, Phosphorus-32, Phosphorus-33, Rhenium-186, Rhodium-106, Rubidium-86, Ruthenium-106, Samarium-153, Scandium-46, Silver-110m, Strontium-89, Strontium-90, Sulfur-35, Technetium-99, Terbium-160, Thulium-170, Tungsten-188, Yttrium-90 and Xenon-133.

The radioactive DNA molecule is preferably selected from the group consisting of a radioisotope, a radioactive DNA or an analog thereof, a radioactive RNA, a radioactive nucleotide and a radioactive oligonucleotide. More preferably, the radioactive molecule is a radioactive oligonucleotide. The oligonucleotide is preferably a 2- to 35-mer oligonucleotide, more preferably an 8- to 20-mer oligonucleotide, and most preferably a 15-mer oligonucleotide, such as disclosed previously (U.S. Pat. No. 5,821,354 and U.S. patent application Ser. No. 09/318,106 filed on May 24, 1999, the entire content of which is hereby incorporated by reference).

Another embodiment of this invention is the use of an antisense DNA molecule consisting of DNA sequences that can alter gene expression. These DNA sequences may be complementary to either the 5′-untranslated region (5′-UTR), the coding region and/or the 3′-untranslated region (3′-UTR) of any targeted gene. The antisense oligonucleotide is preferably a 2- to 50-mer oligonucleotide, more preferably a 12- to 25-mer oligonucleotide, and most preferably a 15 to 20-mer. The hybridization of the antisense oligonucleotide to the target gene sequence is responsible to alter the expression of the said gene and, in consequence, produce the desired therapeutic effect.

Still another embodiment of this invention is the use of a DNA plasmid molecule consisting of DNA sequences that are encoded in a circular fashion. The plasmid may be transferred into the cells of tissues adjacent to the drug-eluting device. Appropriate intracellular enzymes activate the plasmid, inducing the expression of the encoded gene. As a result, the encoded gene will be expressed within the cell, which may then produce the desired therapeutic effect.

In accordance with another aspect of the invention, the method of the present invention is rapid and allows obtaining a radioactively coated artificial occlusion device during the clinical procedure, on which a radioisotope-containing molecule is effectively and uniformly loaded. This contrasts with previous devices disclosed in WO 99/61107, U.S. Pat. No. 5,498,27, U.S. Pat. No. 6,056,686 and U.S. application Ser. No. 09/510,797, whereas the coils are implanted using an ion implantation system in which the coils must be prepared in advance.

The method of the present invention may also be used to embolize blood vessels and/or for treating hypervascular lesions and to decrease blood flow to hypervascular lesions. The present invention may also be used for vascular occlusion of blood vessels within the vascular systems and for endovascular management of arteriovenous malformations (AVMs) and neoplastic lesions when presurgical devascularization is desirable.

The present invention may also be used for artificial embolization of symptomatic carotid cavenous fistulae (CCF).

The present invention may also be used to occlude the blood supply to AVMs and other vascular lesions of the brain, spinal cord and or any vascular territory.

The present invention may further be used for the interventional radiologic management of AVMs, arteriovenous fistulas (AVFs) and other vascular lesions.

In accordance with the present invention, the artificial occlusion device is immersed into a solution containing the biologically active DNA molecule for a period of time. The DNA molecule is then adsorbed onto the surface of the artificial occlusion device. The coil may be coated with a polymer, a protein or any other substance, prior or following adsorption of the radioactive molecule, to either increase the adsorption of the DNA molecule and/or to control the leaching rate of the said DNA molecule from the device. Strong and effective loading of a biologically active DNA molecule such as a radioactive DNA molecule on the surface of the device was obtained by immersion. Furthermore, biologically active DNA molecule such as radioactive DNA molecule is eluted from the device into the adjacent tissue, which is beneficial for preventing recanalisation. The advantage of the method allows preparation of a DNA coated artificial occlusion device to be used for implantation within the aneurysm just moments after its loading with the radioisotope.

Also in one embodiment of the present invention, the artificial occlusion device is loaded with a radioactive DNA molecule that will possess sufficient radioactivity to prevent recanalization and promote neointima formation within the aneurysm. Adequate dosage of radiation to the target tissue will be administered by two mechanisms. The first is the dosage emitted directly from the radioactive artificial occlusion device into the target tissue. The second mechanism involves drug leaching (elution of the radioisotope into adjacent tissues) from the device, which helps attaining the desired dosage of radioactivity to the aneurysm, since the radioactive molecule elutes out of the device and is incorporated into the targeted area.

In another embodiment of the present invention, the artificial occlusion device loaded with either an antisense DNA molecule or a plasmid can leach into adjacent tissues, which can alter gene expression and function in that tissue.

Further in accordance with the present invention, there is provided a method for treating an aneurysm comprising inserting a filling element and a biologically active DNA molecule into a vessel, at least in close proximity of a neck of an aneurysm, the biologically active DNA molecule preventing recanalization and stimulating neointima formation causing obstruction of the neck of the aneurysm and/or filling up the aneurysm.

The present invention allows inhibiting the recanalization process and increasing neointima formation at the neck of an aneurysm and within treated lesions, in order to improve long-term results of endovascular treatment.

The present invention further allows the rapid preparation of occlusion devices and allows obtaining a coating of the active DNA molecule onto the device.

The preparation of the occlusion device can be prepared during the clinical procedure for filling aneurysms.

The preparation can be performed on coils of various diameters and lengths.

By the term artificial occlusion device, it is intended to mean any device used reducing or obstructing the blood flow in a vessel and also any device for the treatment of aneurysms for which leaching of biologically active DNA molecules into adjacent tissues would be beneficial. Such device may be without limitation a coil, preferably a stainless steel, platinum or platinum/tungsten allow coil, a stent, a wire or any other device to which a person of the art may think of for stimulating and increasing neointima formation.

By the term analog of DNA, it is intended to mean nucleic acid sequences such as circular or non-circular double-strand DNA sequences, single-strand DNA sequences, RNA or any combination thereof.

By the term radioactive molecule, it is intended to mean a molecule carrying at least one radioactive element.

By the term antisense oligonucleotide, it is intended to mean nucleic acid sequences that can inhibit gene expression.

By the term plasmid, it is intended to mean DNA sequences encoded in a circulation fashion to induce gene expression.

### BRIEF DESCRIPTION OF THE DRAWINGS

Having thus generally described the nature of the invention, reference will now be made to the accompanying drawings, showing by way of illustration a preferred embodiment thereof, and wherein:

FIG. 1 is partial schematic cross-sectional view of an artery having an aneurysm filled with a drug eluting artificial occlusion coil;

FIG. 2 is a partial schematic representation of a micro catheter provided with a drug eluting artificial occlusion coil in accordance with one embodiment of the invention;

FIG. 3 is an enlarged cross-sectional view taken along the lines 3-3 of FIG. 2;

FIG. 4 illustrates the effect of ³²P-oligonucleotide preparation on efficacy of loading onto an artificial occlusion coil;

FIG. 5 illustrates the effect of temperature on coating an artificial occlusion coil with a radioactive 15-mer oligonucleotide;

FIG. 6 illustrates the effect of increasing concentrations of a radioactive 15-mer oligonucleotide solution on coating onto an artificial occlusion coil;

FIG. 7 is a line graph of a retention profile of ³²P-oligonucleotide coated artificial occlusion coil when exposed to complete culture media;

FIG. 8 is a bar graph illustrating remaining activity onto a coil dipped into a ³²P-oligonucleotide solution following passages into a microcatheter;

FIG. 9 is a bar graph illustrating the effects of sulfuric acid washings of ³²P-oligonucleotide loading and retention onto coils;

FIG. 10 is a line graph of the retention profile of ³²P-oligonucleotide immobilized onto an artificial occlusion coil when deposited into dog arteries in vivo; and

FIG. 11 is a bar graph illustrating ³²P-oligonucleotide leaching into an artery and the thrombus produced when inserting the coil within either the maxillary, cervical or vertebral arteries.

### DETAILED DESCRIPTION OF THE INVENTION

In accordance with one embodiment of the present invention there is provided a DNA eluting artificial occlusion device for treating aneurysm, which would stimulate neointima formation and thus increase neointima formation in aneurysms treated endovascularly. It is known that the mechanism by which bare coils function is to create an intra-aneurysmal thrombus (Casaco et al., J. Neurosurg 79:3-10, 1993), leading to the occlusion of the aneurysm. Since there is lack of smooth muscle cell proliferation and because of the biological evolution of the thrombus, the occluded aneurysm will eventually recanalize, which can ultimately lead to future rupture of the aneurysm. Thus, recanalization of aneurysms has been observed in patients in follow-up angiographies following the procedure (Cognard et al., Radiology 212:348-356, 1999).

Since the coils are inert, a strategy was devised to render them biologically active to prevent aneurysm recanalization. In the field of cardiology, it is known that treatment of the coronary artery with various regimens of radiation inhibits smooth muscle cell proliferation and thus can decrease neointima formation (Walksman, Cardiovasc Radiat Med, 1:20-29, 1999). However, it has been reported that radiation doses insufficient to inhibit smooth muscle cell proliferation can in fact produce the opposite effect, that is stimulate neointima formation (Albiero et al., Circulation 101:2454, 2000). Indeed, experimentally produced radioactive stents, used to prevent restenosis post angioplasty, induces excessive neointima thickening at the stent edges because of insufficient dosing of radiation, creating what is known in the field of cardiology as the edge effect.

Therefore, the present invention take advantage of the edge effect to induce neointima formation that may be promoted by fibrin-thrombus deposition, over-expression of tissue factor, inflammation and growth factor secretion by inflammatory cells, by stimulation of extra cellular matrix by neointimal cells or by any other unknown mechanisms. Thus a radioactive source such as a ³²P-oligonucleotide, delivered directly within the aneurysm at low dose, prevents recanalization and increases neointima formation at the neck and within the aneurysm. This stimulation would then decrease the incidence of recurrence.

It was found that leaching over time of a biologically active DNA in surrounding tissues improves long-term results. Accordingly, there is provided in the present invention a new eluting occlusion device, a method of using it and a new method of preparing it.

In another embodiment, the biologically active DNA such as antisense oligonucleotides or plasmids can alter gene expression in adjacent tissues. In an example involving antisense oligonucleotides, it has been previously reported that an antisense oligonucleotide inhibiting the expression of somatostatin can induce lymphocyte proliferation (Aguila et al., Endocrinology 137(5): 1585-1590, 1996). This localized proliferation of lymphocytes within an aneurysm could initiate biochemical reactions within the aneurysm, which would ultimately lead to an effective treatment of aneurysms. Therefore, in this example, the inhibition of gene expression can induce cellular proliferation, which can ultimately lead to the healing of an aneurysm.

In another example, a plasmid expressing platelet-derived growth factor (PDGF) was transfected into porcine iliofemoral arteries. This elicited intimal thickening of the arteries 21 days following transfection (Nabel et al., J. Clin. Invest., 91(4): 1822-1829, 1993). Although this work was presented for proof of concept of the role of PDGF in restenosis and being an undesired treatment method for that particular pathology, a plasmid inducing cell proliferation in an aneurysm would definitively be desirable. To summarize, delivery of non-radioactive biologically active DNA molecules that can alter gene expression, are potential strategies to improve results of the treatment of aneurysms.

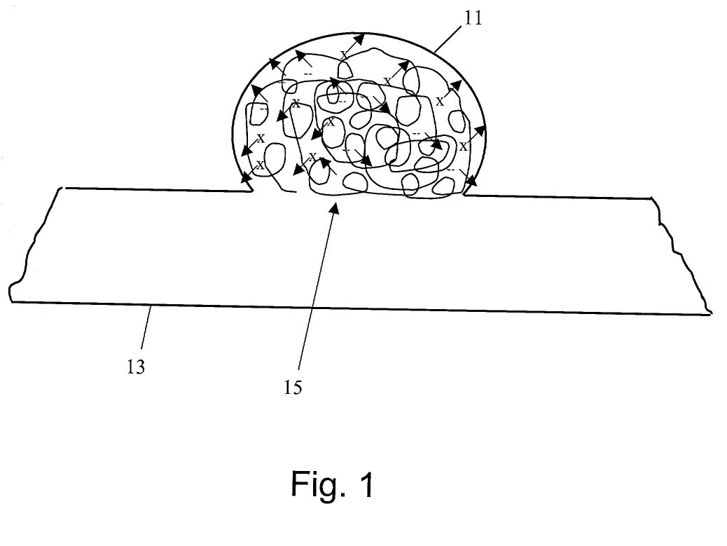

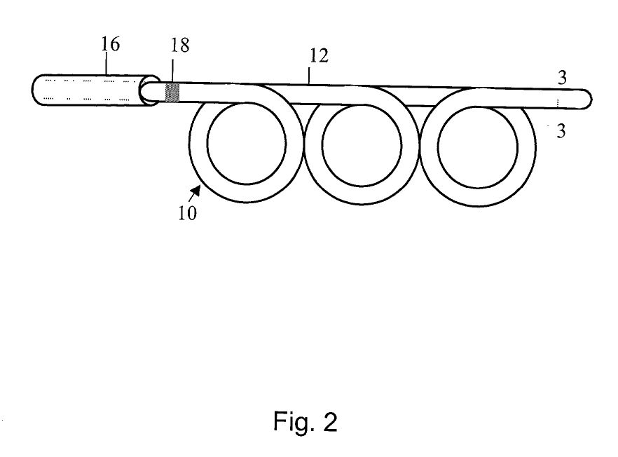

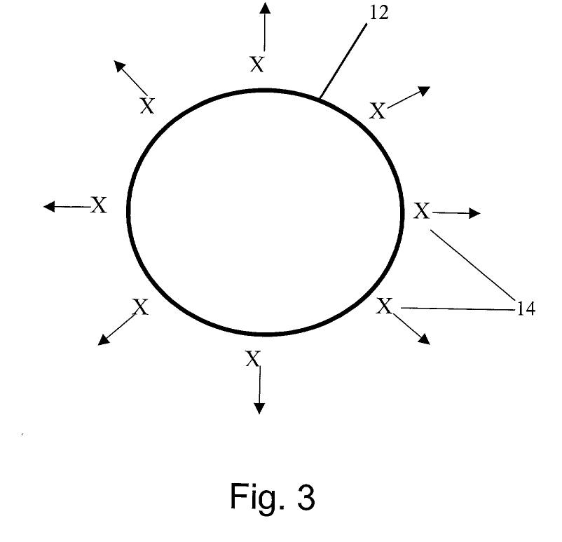

As seen in FIGS. 1 to 3, in accordance with one embodiment of the present invention, there is provided an artificial occlusion device 10 designed for endovascular treatment of an aneurysm 11 located within the vasculature 13, and preferably of an intracranial aneurysm. More than one coil can be placed within an aneurysm, resulting in a mass of coils that seals the aneurysm 15. The coils are delivered to the aneurysm through a catheter 16. However, the artificial occlusion device 10 is not restricted to this use as it could also be used to close any body lumen, such as vascular lumen or others. The artificial occlusion device 10 comprises a detachable filling coil 12, onto which is attached an artificial occlusion DNA eluting coil 14.

One example of such an artificial occlusion device is a Guglielmi detachable coil in which a platinum coil is attached to a stainless steel delivery wire by the use of a junction, which is electrically unstable 18. The stainless steel delivery wire is then attached to an electrode, more particularly an anode, while another electrode, and more particularly a ground or cathode, is attached to the body. Both electrodes, cathode and anode, are then attached to a current generator, such as a battery-operated unit, and a low current is applied to the delivery wire. This causes the electrically susceptible junction to dissolve, releasing the platinum coil from its delivery wire. The current may be applied to the coils for a certain period of time until it finally dissolves.

In this embodiment, the embolic agent is a detachable coil coated with a biologically active DNA, preferably a platinum coil coated with a DNA molecule containing a radioactive source of ³²P, a β-emitting isotope of phosphorus. Accordingly, there is provided in the present invention a new eluting occlusion device, a method of using it and a new method of preparing it. The preparation of the radioactive DNA is a 2-step process. The first step is the synthesis of an internally labeled oligonucleotide, which has been previously disclosed in U.S. Pat. No. 5,821,354. The second step is the purification process of the radioactive oligonucleotide. Following synthesis, the radioactive oligonucleotide was purified on a HPLC system on a Oligo R3 reverse phase column (Perseptive Biosystems, MA) using a 4 solvent gradient composed of the following solvents: A: 0.12 M Glacial Acetic acid—0.16 M triethylamine; B: 80% Acetonitrile—20% water; C: 3% trifluoroacetic acid (TFA); and D: bidistilled water. The oligonucleotide was purified using the multi-solvent step gradient illustrated in Table 1.

TABLE 1
Gradient used for oligonucleotide purification.

| Step | Time | | | | | | |
|---|---|---|---|---|---|---|---|
| # | (min) | Flow | % A | % B | % C | % D | Comments |
| 1 | 0 | 5 | 85 | 15 | 0 | 0 | Elimination of failure |
| 2 | 3 | 5 | 85 | 15 | 0 | 0 | sequences |
| 3 | 4 | 5 | 0 | 0 | 0 | 100 | Buffer Wash |
| 4 | 7.5 | 5 | 0 | 0 | 0 | 100 | Cleavage of DMT |
| 5 | 8.5 | 5 | 0 | 0 | 100 | 0 | |
| 6 | 9 | 5 | 0 | 0 | 100 | 0 | TFA Wash |
| 7 | 15 | 5 | 0 | 0 | 0 | 100 | |
| 8 | 18 | 5 | 0 | 0 | 0 | 100 | Collect Sample |
| 9 | 21 | 5 | 0 | 20 | 0 | 80 | |
| 10 | 22 | 5 | 0 | 20 | 0 | 80 | |
| 11 | 25 | 5 | 0 | 100 | 0 | 0 | Column Wash |
| 12 | 26 | 5 | 0 | 100 | 0 | 0 | |
| 13 | 31 | 5 | 85 | 15 | 0 | 0 | Equilibrate |
| 14 | 31.1 | 0 | 85 | 15 | 0 | 0 | Column |

The first step was to eliminate the failure sequences from the final product. Only the final product bears the DMT (dimethoxytrityl) moiety, which will remain in the reverse phase column. This step will be followed by a washing step to desalt the oligonucleotide. The next step involves elimination of the DMT moiety by briefly exposing the oligonucleotide to trifluoroacetic acid (TFA). The TFA is washed and a gradient is then applied to elute the purified oligonucleotide at approximately 18 to 19 minutes. Then, the column is washed and equilibrated for the next run. Following purification, the dilute oligonucleotide solution is then placed in an evaporator for a period of time ranging from 6 to 18 hours. The oligonucleotide pellet is then suspended in a small volume of water during 2 hours. When diluted to the appropriate concentration, the solution is then heated at 65° C. before performing immobilization of the DNA onto the occlusion device.

In one embodiment of the invention, an artificial occlusion device is dipped into a solution containing a ³²P-oligonucleotide for a period of time of approximately 15 minutes followed by a washing step in an appropriate media, such as water or phosphate buffered saline. The ³²P-oligonucleotide may be substituted to an antisense DNA molecule.

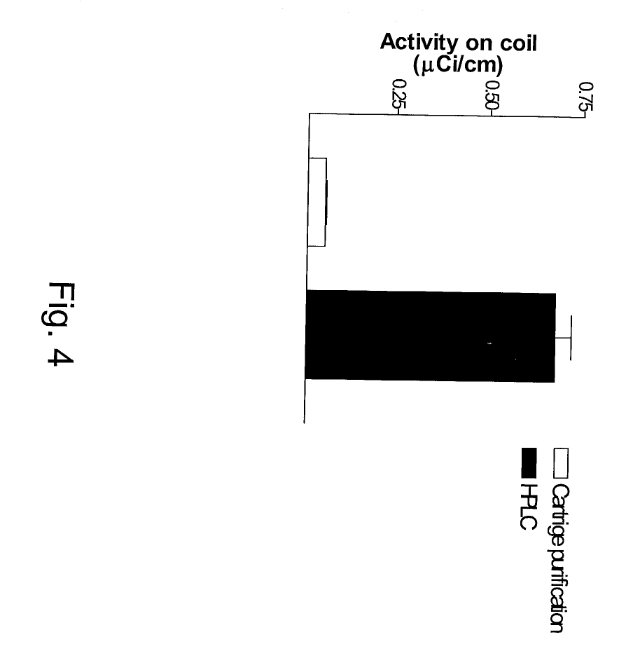

The ³²P-oligonucleotide is then adsorbed onto the surface of the artificial occlusion device, yielding effective loading of ³²P-oligonucleotides on the metallic surface of the device. As illustrated in FIG. 4, it was essential that the DNA be purified by HPLC before immobilization. If the DNA is only passed through a purification cartridge for desalting purposes only, the radioactive DNA will not be deposited onto the occlusion device. Therefore, this demonstrates that HPLC purification of the oligonucleotide is essential to the success of the entire DNA depositing process onto the occlusion device.

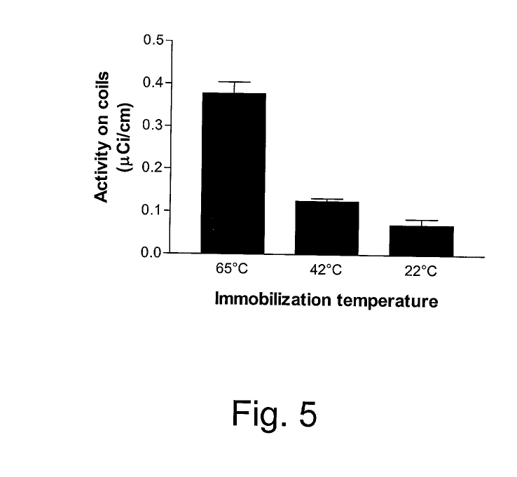

As illustrated in FIG. 5, levels of radioactivity adsorbed onto the coil are in function of temperature. It was observed that binding of the ³²P-oligonucleotide is increased when the temperature of the radioactive solution is at 65° C., compared to 22° C. and 42° C.

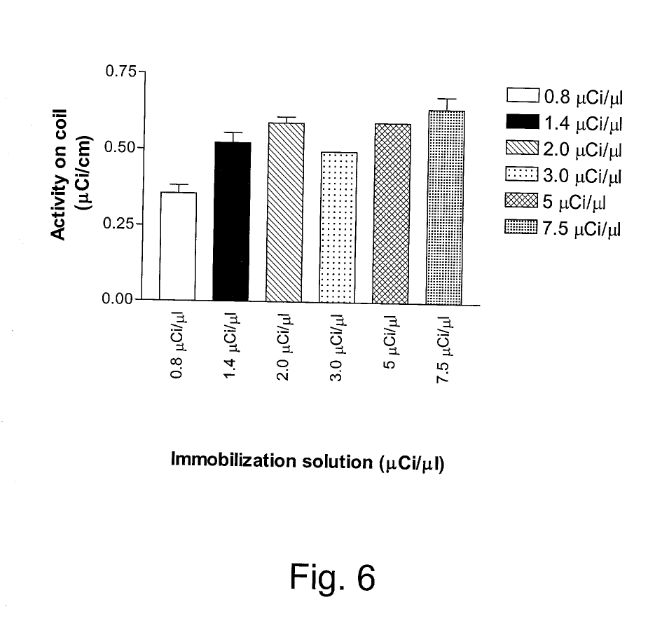

FIG. 6 illustrates the increases of adsorption of ³²P-oligonucleotide onto a coil. When the coils were exposed to 100 μl of radioactive DNA solution containing 0.8 to 7.5 μCi/μl, coils with activities varying from 0.3 to 0.7 μCi/cm were obtained. Following loading, the radioactive eluting coils were placed in a biological medium composed of DMEM supplemented with 20% Fetal Bovine Serum (FBS, Gibco) at 37° C. with constant agitation. The coils were taken out of the media for assessment of radioactivity levels then placed in fresh media at the following incubation times: 1 h, 4 h, 1, 2, 4, 6 and 8 days.

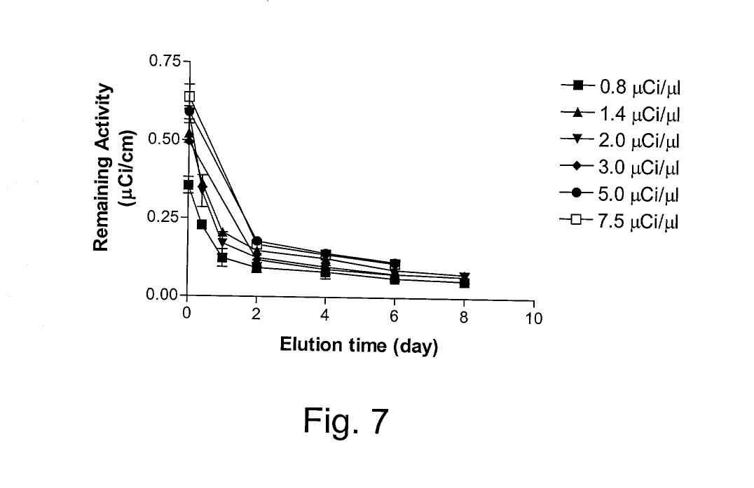

FIG. 7 illustrates the retention profile of coated ³²P-oligonucleotide onto the artificial occlusion coil in a biological medium when initially exposed to activities of 0.8 to 7.5 μCi/μl. As illustrated in FIG. 7, following incubation of the artificial occlusion coil at 37° C., the residual activity on coils decreased as a function of time. After 6 days of incubation, the remaining activity on the coils varied from 6% to 21% for all conditions. It should be noted that the bulk of the drop of activity occurs during the first 4 hours of elution.

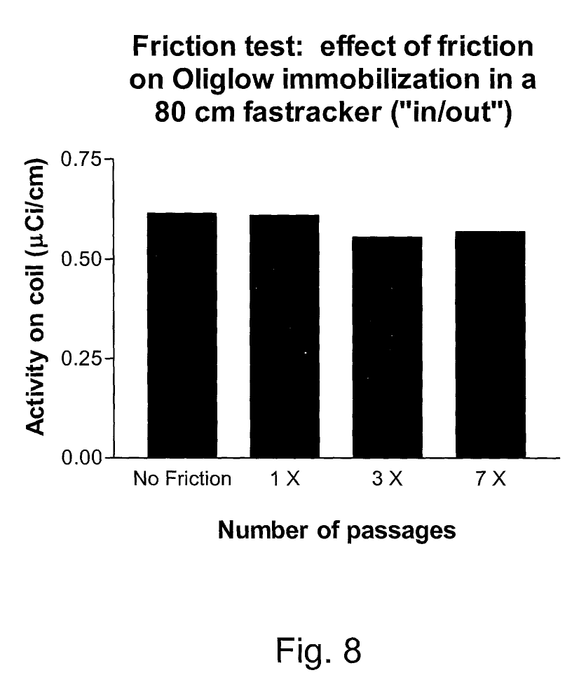

Immediately following deposition of a radioactive DNA molecule onto a coil, a friction test was performed to assess whether the ³²P-oligonucleotide would be released from the coil by the friction in the microcatheter. This would mimic the intervention in which the operator inserts the coil into a microcatheter for final placement into the aneurysm. FIG. 8 illustrates the level of radioactivity remaining on a full length GDC-18 soft 3 mm×8 cm coil following repeated insertion into a Fastracker (in/out) microcatheter. There is no significant loss of radioactivity even when the coil is inserted and removed 7 times from the microcatheter. It is concluded that the ³²P-oligonucleotide is bound onto the platinum coil and that little activity was lost due to the passage through the catheter. Therefore, the coil coated with biologically active DNA looses little DNA until it is in place in the aneurysm.

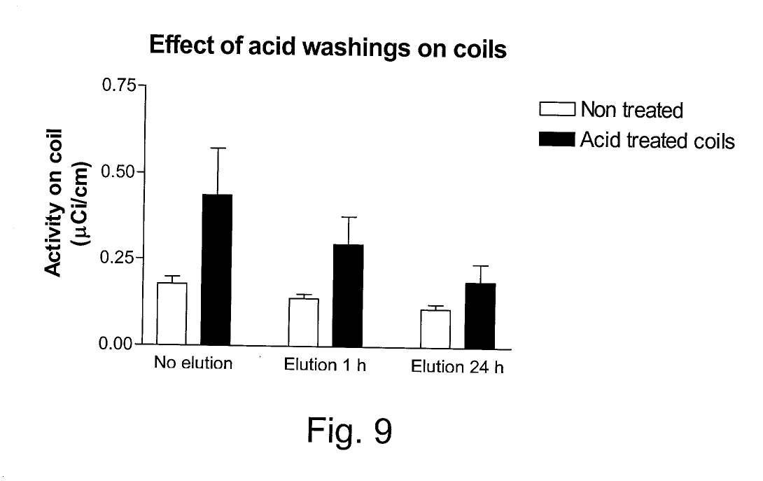

³²P-oligonucleotide binding to platinum may be affected by contamination of the surface of the coils. The effect of a “surface preparation” was investigated in order to minimize the level of potential contamination of the surface of the coils and its impact on ³²P-oligonucleotide binding to the coils. It is known that sulfuric acid removes all residues residing on the surface of metals, such as carbon-based molecules (oils, carbon monoxide, carbon dioxide) and other types of impurities. A comparison of the deposition of ³²P-oligonucleotide onto non-treated coils versus coils exposed to sulfuric acid for 2 hours was performed. ³²P-oligonucleotide was more readily bound to the coils that were acid-washed than the non-treated coils (FIG. 9). The observed increase in activity was retained by the coils following 1 and 24 hours of elution. Thus, sulfuric acid treatment of coils increases the loading of biologically active DNA molecules.

X-ray photoelectron spectroscopy (XPS) scanning of coils showed in all cases the presence of carbon, oxygen, platinum and tungsten. Table 2 summarizes the mean±SEM of 4 distinct measurements from 2 coils from 2 different lots. Approximately 45% of the surface of an untreated coil is covered by a carbon-containing molecule, while 27% of the surface is composed of oxygen. Surprisingly, only 22% of the surface is platinum and 5% tungsten. The fine spectrometry of the carbon present on the coils suggests that it is present mostly (>90%) in the form of a carbon-carbon bond, with the remainder being a carbon-oxygen bond. This means that the coils are covered with a carbon-based molecule that can range from an aliphatic moiety to an aromatic compound.

TABLE 2
Effect of sulfuric acid treatment on
the chemical composition of coils

| | Tungsten | Platinum | Carbon | Oxygen |
|---|---|---|---|---|
| | (W) | (PT) | (C) | (O) |
| Non-treated | 5.3 ± 0.53 | 22.53 ± 0.76 | 44.75 ± 0.74 | 27.33 ± 1.62 |
| coils | | | | |
| Sulfuric acid | 5.33 ± 0.35 | 29.25 ± 1.10 | 35.95 ± 0.7 | 29.4 ± 0.91 |
| treated coils | | | | |
| Student's t | NS | P < 0.0024 | P < 0.0001 | NS |
| test | | | | |

Values are the mean ± SEM of 4 determinations from 2 coils of different lots

Sulfuric acid treatment of the coils for 2 hours decreased the impurity content of the coils, increasing ³²P-oligonucleotide binding efficiency. Thus, it was concluded that manufactured coils contain surface bound molecules that hinder the binding of the ³²P-oligonucleotide. Therefore, if substantial increases of DNA loading is required on the coils, an option would be to pre-treat the coils with for example sulfuric acid. This could be accomplished by including a washing step in the manufacturing process of the coils.

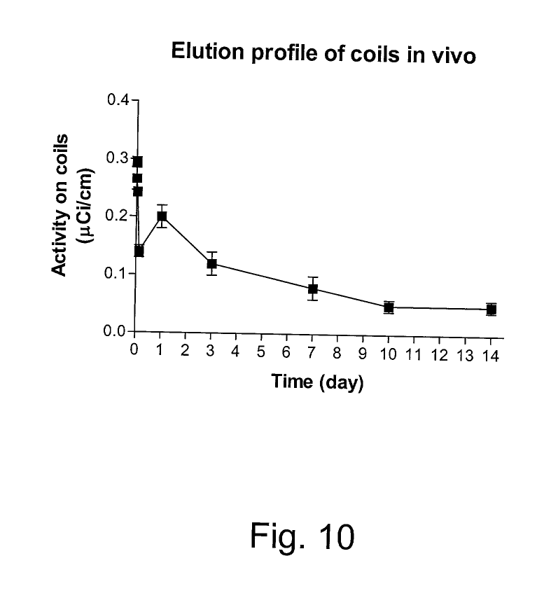

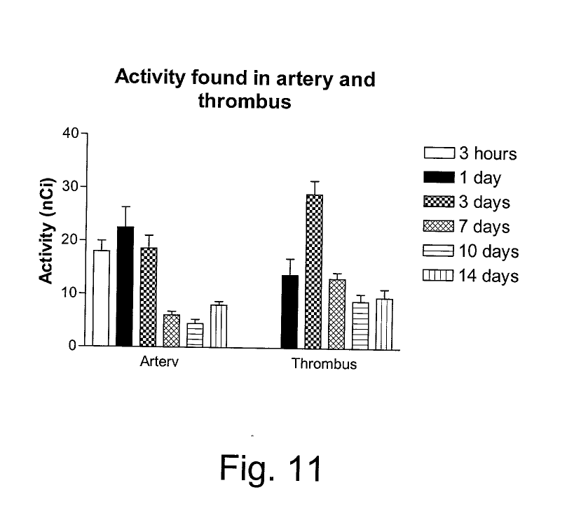

Since satisfactory loading onto the coils and elution profiles in biological medium were obtained, elution profiles of the ³²P-oligonucleotide from the coils in dogs and incorporation in the adjacent tissues were then assessed in vivo (FIGS. 10 and 11). Leaching of the ³²P-oligonucleotide into the adjacent tissues was investigated. To perform this experiment, six healthy Beagle dogs weighing 15-20 kg were anesthetized according to standard procedures. A percutaneous femoral puncture was used to reach the aorta and bilateral maxillary, cervical and vertebral arteries with 2F microcatheters introduced coaxially through 5F catheters. A platinum coil (Guglielmi Detachable Coils, GDC, 3 mm in diameter, 8 cm in length) was dipped into a ³²P-oligonucleotide solution (0.8 μCi/μl) that produced coils with activities of 0.294±0.009 μCi/cm (n=57). Following placement of the coils in the arteries or the aorta for their respective times as shown in FIGS. 10 and 11, the arteries containing the coils and thrombus were then harvested from the animal. Radioactivity levels of the coils were assessed directly by scintillation counting (FIG. 10) while the artery and the thrombus were dissolved in triethylamine hydroxide then submitted to scintillation counting (FIG. 11).

FIG. 10 illustrates the activities of the ³²P-oligonucleotide-coated coils as a function of time. The 5 and 60 min time points were obtained by exposing the coils within the dog aorta, producing activities of 0.27±0.02 (n=7) and 0.24±0.03 (n=7), respectively. ³²P-oligonucleotide radiolabeled coils were then inserted in maxillary, vertebral or cervical arteries for 3 hours, 1, 3, 7, 10 or 14 days.

FIG. 11 illustrates leaching of the ³²P-oligonucleotide into the adjacent artery and thrombus for the 3 hours, 1, 3, 7, 10 or 14 days incubation. Activities up to 30 nCi were found into the adjacent artery and thrombus. The leaching of the ³²P-oligonucleotide into adjacent tissues is an advantage compared to permanent retention of radioactivity onto coils, since the activity diffused into the thrombus and artery prevents recanalization at some distance from the coil surface.

The simplicity of the method of the present invention allows preparation of the artificial occlusion device coated with biologically active DNA molecules to be used for implantation shortly before the implantation procedure. When radioactive DNA molecules are involved, other γ and β emitters such as rhenium, strontium or any other radioactive source can be used for the same purpose.

In use, the filling coil 12 and the β-emitting radioactive source 14 are delivered with a microcatheter 16. For filling the aneurysm 11 with the artificial occlusion device 10, the same procedure is done as is presently being done for filling any aneurysm. The difference being that currently, aneurysms are being filled with a filling coil alone, whereas in accordance with the present invention, aneurysms treated with the present invention would be filled with a filling coil coated with at least one biologically active DNA molecule that will slowly release within the aneurysm either a β-emitting source or a gene altering DNA analog.

Briefly, the microcatheter 16 is brought to the aneurysm 11 to be treated from within a blood vessel 13. The filling coil 12 pushed in the aneurysm 11 will release the biologically active DNA molecule. Sufficient filling coil 12 is inserted in the aneurysm 11 for filling and packing it. The β-emitting radioactive molecule, which is eluted within the aneurysm, prevents recanalization and stimulates neointima formation, which will cause the closing of the neck of the aneurysm 11 therefore repairing the blood vessel.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

What is claimed is:

1. A method for reducing or blocking blood flow in a vessel, said method comprising the step of introducing at a desired site in the vessel a slow-releasing, biologically active DNA molecule-leaching device, said device releasing a biologically active DNA molecule at the site for stimulating neointima formation and increasing neointima thickness, said neointima formation reducing or blocking the blood flow in the vessel, said biologically active DNA molecule released at the site is absorbed by surrounding tissues of the vessel for providing long-term vascular reduction or obstruction of the blood flow in the vessel.

2. The method of claim 1, where the device comprises a detachable filling element and said biologically active DNA molecule comprising at least one radioactive source, said detachable filling element and said radioactive source being adapted to be inserted into the vessel at the desired site, said radioactive source stimulating neointima formation for providing long-term vascular reduction or obstruction of the blood flow in the vessel.

3. The method of claim 2, where the detachable filling element is a coil.

4. The method of claim 3, wherein the coil is a platinum coil, a platinum/tungsten alloy coil or a stainless steel coil.

5. The method of claim 1, wherein the radioactive source is a β-emitting source.

6. The method of claim 1, wherein the radioactive source comprises a polymer and a radioisotope.

7. The method of claim 5, wherein the β-emitting source is at least one β-emitting source from Antimony-124, Cesium-134, Cesium-137, Calcium-45, Calcium-47, Cerium 141, Chlorine-36, Cobalt-60, Europium-152, Gold-198, Hafnium-181, Iodine-131, Iridium-192, Iron-59, Lutetium-177, Mercury-203, Neodymium-147, Nickel-63, Phosphorus-32, Phosphorus-33, Rhenium-186, Rubidium-86, Ruthenium-106, Samarium-153, Scandium-46, Silver-110m, Strontium-89, Strontium-90, Sulfur-35, Technetium-99, Terbium-160, Thulium-170, and Yttrium-90.

8. The method of claim 1, wherein the occlusion device is introduced in the vessel at the site concurrently with a polymer for filling the vessel.

9. The method of claim 1, wherein the biologically active DNA molecule is an antisense oligonucleotide stimulating cell proliferation for for providing long-term vascular reduction or obstruction of the blood flow in the vessel.

10. The method of claim 1, wherein the biologically active DNA molecule is a plasmid stimulating cell proliferation for providing long-term vascular reduction or obstruction of the blood flow in the vessel.

11. A method for sustained vascular occlusion of a blood vessel, said method comprising the step of introducing at a site in the vessel a slow-releasing, biologically active DNA molecule-leaching device, said device releasing a biologically active DNA molecule at the site for stimulating neointima formation and increasing neointima thickness, said neointima formation filling the vessel, said biologically active DNA molecule released at the site is absorbed by surrounding tissues of the vessel for providing long-term vascular occlusion of the blood vessel and preventing recanalisation.

12. The method of claim 11, where the device comprises a detachable filling element and said biologically active DNA molecule comprising at least one radioactive source, said detachable filling element and said radioactive source being adapted to be inserted into the vessel at a desired site, said radioactive source stimulating neointima formation for providing long-term vascular occlusion of the blood vessel.

13. The method of claim 12, where the detachable filling element is a coil.

14. The method of claim 13, wherein the coil is a platinum coil, a platinum/tungsten alloy coil or a stainless steel coil.

15. The method of claim 11, wherein the radioactive source is a β-emitting source.

16. The method of claim 11, 12, 13, 14 or 15, wherein the radioactive source comprises a polymer and a radioisotope.

17. The method of claim 15, wherein the β-emitting source is at least one β-emitting source from Antimony-124, Cesium-134, Cesium-137, Calcium-45, Calcium-47, Cerium 141, Chlorine-36, Cobalt-60, Europium-152, Gold-198, Hafnium-181, Iodine-131, Iridium-192, Iron-59, Lutetium-177, Mercury-203, Neodymium-147, Nickel-63, Phosphorus-32, Phosphorus-33, Rhenium-186, Rubidium-86, Ruthenium-106, Samarium-153, Scandium-46, Silver-110m, Strontium-89, Strontium-90, Sulfur-35, Technetium-99, Terbium-160, Thulium-170, and Yttrium-90.

18. The method of claim 11, wherein the occlusion device is introduced in the vessel at the site concurrently with a polymer for filling the vessel.

19. The method of claim 11, wherein the biologically active DNA molecule is an antisense oligonucleotide stimulating cell proliferation for filling up the vessel and preventing recanalization thereof.

20. The method of claim 11, wherein the biologically active DNA molecule is a plasmid stimulating cell proliferation for filling up the vessel and preventing recanalization thereof.

21. A method for sustained treatment of an aneurysm, said method comprising the step of introducing at an aneurysm site a slow-releasing, biologically active DNA molecule-leaching device, said device releasing a biologically active DNA molecule at the aneurysm site for stimulating neointima formation and increasing neointima thickness, said neointima formation filling the aneurysm, said biologically active DNA molecule released in the aneurysm is absorbed by surrounding tissues of the aneurysm for providing long-term treatment of said aneurysm and preventing recanalisation.

22. The method of claim 21, where the device comprises a detachable filling element and said biologically active DNA molecule comprising at least one radioactive source, said detachable filling element and said radioactive source being adapted to be inserted into a vessel at least in close proximity of a neck of an aneurysm, said radioactive source stimulating neointima formation for obstructing the neck of the aneurysm or filling up the aneurysm.

23. The method of claim 22, where the detachable filling element is a coil.

24. The method of claim 23, wherein the coil is a platinum coil, a platinum/tungsten alloy coil or a stainless steel coil.

25. The method of claim 21, wherein the radioactive source is a β-emitting source.

26. The method of claim 21, 22, 23, 24 or 25, wherein the radioactive source comprises a polymer and a radioisotope.

27. The method of claim 25, wherein the β-emitting source is at least one β-emitting source from Antimony-124, Cesium-134, Cesium-137, Calcium-45, Calcium-47, Cerium 141, Chlorine-36, Cobalt-60, Europium-152, Gold-198, Hafnium-181, Iodine-131, Iridium-192, Iron-59, Lutetium-177, Mercury-203, Neodymium-147, Nickel-63, Phosphorus-32, Phosphorus-33, Rhenium-186, Rubidium-86, Ruthenium-106, Samarium-153, Scandium-46, Silver-110m, Strontium-89, Strontium-90, Sulfur-35, Technetium-99, Terbium-160, Thulium-170, and Yttrium-90.

28. The method of claim 21, wherein the occlusion device is introduced in the aneurysm site concurrently with a polymer for filling the aneurysm site.

29. The method of claim 21, wherein the biologically active DNA molecule is an antisense oligonucleotide stimulating cell proliferation for filling up the aneurysm and preventing recanalization of the aneurysm.

30. The method of claim 21, wherein the biologically active DNA molecule is a plasmid stimulating cell proliferation for filling up the aneurysm and preventing recanalization of the aneurysm.

31. A method for sustained treatment of a hypervascular lesion, said method comprising the step of introducing in the vessel feeding the lesion a slow-releasing, biologically active DNA molecule-leaching device, said device releasing a biologically active DNA molecule in the lesion for stimulating neointima formation and increasing neointima thickness, said neointima formation reducing or blocking blood flow in the vessel at the lesion, said biologically active DNA molecule released at the lesion is absorbed by surrounding tissues of the vessel for providing long-term vascular reduction or blocking of the blood flow in the vessel.

32. The method of claim 31, where the device comprises a detachable filling element and said biologically active DNA molecule comprising at least one radioactive source, said detachable filling element and said radioactive source being adapted to be inserted into the vessel at the lesion, said radioactive source stimulating neointima formation for providing long-term vascular reduction or blocking of the blood flow in the vessel.

33. The method of claim 32, where the detachable filling element is a coil.

34. The method of claim 33, wherein the coil is a platinum coil, a platinum/tungsten alloy coil or a stainless steel coil.

35. The method of claim 31, wherein the radioactive source is a β-emitting source.

36. The method of claim 31, 32, 33, 34 or 35, wherein the radioactive source comprises a polymer and a radioisotope.

37. The method of claim 35, wherein the β-emitting source is at least one β-emitting source from Antimony-124, Cesium-134, Cesium-137, Calcium-45, Calcium-47, Cerium 141, Chlorine-36, Cobalt-60, Europium-152, Gold-198, Hafnium-181, Iodine-131, Iridium-192, Iron-59, Lutetium-177, Mercury-203, Neodymium-147, Nickel-63, Phosphorus-32, Phosphorus-33, Rhenium-186, Rubidium-86, Ruthenium-106, Samarium-153, Scandium-46, Silver-110m, Strontium-89, Strontium-90, Sulfur-35, Technetium-99, Terbium-160, Thulium-170, and Yttrium-90.

38. The method of claim 31, wherein the occlusion device is introduced in the vessel at the lesion concurrently with a polymer for filling the vessel.

39. The method of claim 31, wherein the biologically active DNA molecule is an antisense oligonucleotide stimulating cell proliferation for reducing or blocking the blood flow in the vessel.

40. The method of claim 31, wherein the biologically active DNA molecule is a plasmid stimulating cell proliferation for reducing or blocking the blood flow in the vessel.

41. A method for preparing a DNA leaching artificial occlusion device, said method comprising the step of providing a solution of an HPLC-purified DNA and dipping an artificial occlusion device in said solution for adsorbing DNA onto the occlusion device in such a manner that said DNA leaches from said occlusion device.

42. The method of claim 41, wherein the HPLC-purified DNA transiently contains dimethoxytrityl (DMT) moiety.

43. The method of claim 42, further comprising before the step of dipping the occlusion device in the solution, a step of desalting the HPLC-purified DNA.

44. The method of claim 41, wherein the solution of HPLC-purified DNA is heated above 65° C. before dipping the occlusion device therein.
